(19) United States
(12) Patent Application Publication
(10) Pub. No.: US 2009/0030470 A1
(43) Pub. Date: Jan. 29, 2009

(54) IMPLANTABLE HEART STIMULATION DEVICE WITH REMEDIAL RESPONSE TO ANODAL CAPTURE

(76) Inventors: Nils Holmstrom, Jarfalla (SE); Anders Bjorling, Solna (SE)

Correspondence Address: SCHIFF HARDIN, LLP;PATENT DEPARTMENT, 6600 SEARS TOWER, CHICAGO, IL, 60606-6473, US

(21) Appl. No.: 12/160,849

(22) PCT Filed: Jan. 26, 2006
(86) PCT No.: PCT/SE2006/000119

§ 371 Date: Oct. 1, 2008

(51) Int. Cl. A61N 1/368

(52) U.S. Cl. 607/27

(57) ABSTRACT

An implantable heart stimulating device has a left ventricular coronary sinus electrode lead provided with a tip electrode, a right ventricular electrode lead provided with a ring electrode, and a pulse generator connected to the leads that applies stimulation pulses between the tip electrode and the ring electrode, with the tip electrode serving as the anode. A monitoring unit monitors for and detects anodal capture at the right ventricular ring electrode subsequent to a stimulation. If anodal capture is detected, either a threshold search is performed by varying the pulse width and/or pulse amplitude of stimulation pulses in order to identify stimulation pulse characteristics that avoid anodal capture at the ring electrode, or at least one further electrode is activated to function as an indifferent electrode together with the ring electrode, also in order to avoid anodal capture at the ring electrode.

### BACKGROUND OF THE INVENTION

1. Field of the Invention

The present invention relates to an implantable heart stimulating device, and in particular to a biventricular implantable heart stimulating device with which left ventricular (LV) stimulation is performed between an LV-tip electrode, being the cathode, and a right ventricular (RV) ring electrode, being the anode.

2. Description of the Prior Art

When stimulating LV-tip to RV-ring in a biventricular system a so called anodal stimulation generating an anodal capture may occur on the RV-ring. If the left ventricle is stimulated first which it often is-both ventricles will depolarize at the same time and a ventricle-ventricle (VV) delay optimization is then not possible to perform.

Furthermore, an automatic capture algorithm may detect loss of capture at each RV stimulation since the RV has already been stimulated and is thus refractory. This, in turn, will lead to unnecessarily going into high output mode and incorrect diagnostics.

As will be discussed in detail below the above problem is related to that the unipolar voltage strength-duration curves for the LV tip and the RV ring electrodes have different shapes. Anodal thresholds are normally higher than cathodal thresholds for the same electrode. The LV thresholds are normally higher than RV thresholds and the ring thresholds are normally higher than the tip thresholds because of different surface area and distance to excitable tissue.

All these circumstances influence the bifocal stimulation thresholds so that anodal threshold may be higher than cathodal at wide pulse width, while the cathodal threshold may be higher for a short pulse width.

In order to fully explain the present invention, a general background will be given in the following.

In order to excite the left ventricle, the lead must be disposed near the left ventricle, preferably in the region of the free lateral or posterior wall, which may most easily be accomplished by placing the lead through the coronary sinus and into a left cardiac vein. Unlike a lead for the right ventricle, which is disposed within the ventricle where a tip electrode can be fixed into the myocardium, the electrodes of a lead in a cardiac vein cannot be fixed into the myocardium since that would require puncturing the vein. Instead, in the case of a bipolar lead, both the tip and ring electrodes (or proximal and distal electrodes in the case where both electrodes are ring electrodes or other types of structures) are positioned within the vein adjacent to the left ventricular myocardium. Because the surface area of the tip electrode is smaller than the area of the ring electrode, the current density will be higher at the tip electrode and, thus, the threshold lower. Normally, therefore, the tip of a bipolar lead is used as the cathode in order to achieve the desirable cathodal capture when a voltage pulse is impressed across the two electrodes. (Cathodal capture means that cathodal stimulation is responsible for the contraction.) With a bipolar lead in a cardiac vein, however, both electrodes are external to the myocardium and may have similar capture thresholds so that either anodal or cathodal capture can occur when a pacing pulse is output through the lead. A problem arises when the pulse energy for a bipolar lead in a cardiac vein is adjusted. When the lead is implanted, the capture threshold for the tip or distal electrode (i.e., the electrode usually selected to function as the cathode) may be higher than that of the ring or proximal electrode. When the clinician then determines the capture threshold of the lead with a bipolar pulse in order to adjust the stimulus pulse energy, it is impossible to distinguish between anodal and cathodal capture. There is then a risk that the stimulus pulse energy will be set to an anodal capture threshold when the cathodal capture threshold is higher. As the anodal capture threshold increases over time, the stimulus pulses may no longer be of sufficient energy to excite the left ventricle (diminishing or eliminating the programmed safety margin), and the patient may experience sporadic or total loss of resynchronization therapy.

U.S. Pat. No. 6,687,545 relates to a cardiac stimulation system and method for performing automatic capture verification during bipolar stimulation by eliminating capture verification during a cardiac cycle in which anodal stimulation is detected.

Anodal stimulation is detected by the absence of a delay between the bipolar stimulation pulse and an evoked response sensed at the electrode functioning as the anode during stimulation.

Automatic capture verification during bipolar stimulation is recommended only if anodal stimulation is not detected at a working stimulation output. During automatic capture verification, if anodal stimulation is detected, a capture threshold search is performed. In U.S. Pat. No. 6,687,545 unipolar sensing is performed using e.g. the right ventricular ring electrode and the housing to determine if a stimulation pulse produced anodal stimulation at the ring electrode.

According to the US-patent this is performed by determining the time from the stimulation pulse to the onset of the evoked response.

Typically, a 20 to 40 ms conduction delay to the unipolar ring evoked response signal occurs when only cathodal stimulation is present. Therefore, if there is a delay to the evoked response as determined then anodal stimulation is not indicated and will not interfere with evoked response detection during bipolar evoked response sensing of the bipolar stimulation at the currently programmed output.

If no delay to the evoked response is measured then anodal stimulation is occurring at the ring electrode at the programmed stimulation output.

Thus, the system and method disclosed in U.S. Pat. No. 6,687,545 may be used to verify anodal stimulation.

U.S. Pat. No. 6,421,564 relates to bi-chamber pacing system employing unipolar left heart chamber lead in combination with bipolar right chamber lead. The object is to achieve a system where the left ventricle pacing pulse in a left ventricular pacing vector is directed such that the vector traverses as great a bulk of the left ventricular myocardial mass as possible.

U.S. Pat. No. 6,611,712 relates to an apparatus and method for testing the capture threshold of a bipolar lead of a cardiac rhythm management device in order to determine an appropriate stimulus pulse energy for the lead and/or select an appropriate stimulation configuration.

In accordance with the present invention, two main reasons are identified for the stimulation set-up where the stimulation pulse is applied between a left ventricular (LV) coronary sinus (CS) lead tip electrode, being the cathode, and a right ventricular (RV) ring electrode.

The first reason is to avoid stimulation of nervus pherenicus.

The tip electrode of an LV CS electrode lead is often positioned in close proximity of the nervus pherenicus, which is a nerve that controls the contraction of the diaphragm. It has been found that a direction of the electrical stimulation vector, resulting from a stimulation pulse from an electrode close to nervus pherenicus, that essentially encompasses the nerve, may result in a nerve stimulation that in turn may cause diaphragm contractions. That may especially occur when a bipolar LV CS electrode lead is used, i.e. a ring electrode of the LV CS lead as indifferent electrode (anode).

By using the ring electrode of an RV electrode lead, the electrical stimulation vector then is directed away from nervus pherenicus thus avoiding stimulated diaphragm contractions.

Another reason is that if unipolar stimulation is applied using the housing (case) of the implantable device as indifferent electrode to a LV CS tip electrode, unwanted pocket stimulation may occur, i.e. anodal stimulation at the indifferent case electrode.

Thus, an object of the present invention is to address the situation that occurs when anodal stimulation is detected at an RV ring electrode during a biventricular stimulation mode between an LV CS lead electrode and an RV lead electrode in order to achieve safe and reliable performance of the implantable stimulation device.

### SUMMARY OF THE INVENTION

The above object is achieved in accordance with the present invention by an implantable heart stimulating device having a left ventricular (LV) coronary sinus (CS) electrode lead that carries a tip electrode, a right ventricular (RV) electrode lead that carries a ring electrode, and a pulse generator connected to these leads that applies a stimulation pulse between the tip electrode and the ring electrode, with the tip electrode serving as the cathode. A monitoring unit monitors for and detects anodal capture at the RV ring electrode subsequent to a stimulation. If anodal capture at the RV ring electrode is detected, either a threshold search is performed by varying the pulse width and/or pulse amplitude of the stimulation pulses in order to identify stimulation pulse characteristics that avoid anodal capture at the ring electrode, or at least one further electrode is employed as an indifferent electrode together with the ring electrode, also to avoid anodal capture at the ring electrode.

### BRIEF DESCRIPTION OF THE DRAWINGS

FIG. 1 illustrates two graphs that show strength duration curves in two different situations.

FIG. 2 is a block diagram illustrating a preferred embodiment of the present invention.

### DESCRIPTION OF THE PREFERRED EMBODIMENTS

As stated above the present invention concerns an implantable heart stimulating device, in particular a biventricular pacemaker system, where LV stimulation is performed between the LV-tip (cathode) and the RV-ring (anode).

In a bi-ventricular pacing system, a small diameter, often unipolar, left ventricular (LV), coronary sinus (CS) electrode lead and a bipolar right ventricular (RV) endocardial electrode lead are preferably employed to provide left and right heart chamber pacing/sensing electrodes. The LV CS lead is advanced through the superior vena cava, the right atrium, the ostium of the coronary sinus (CS), the CS, and into the coronary vein descending from the CS to locate the LV active pace/sense electrode at a desired LV pace/sense site.

The RV electrode lead is advanced into the RV chamber to locate RV tip and ring electrodes therein.

A requirement that makes non-simultaneous biventricular pacing and VV-delay optimization is that no anodal ring stimulation capturing the RV-ring is present.

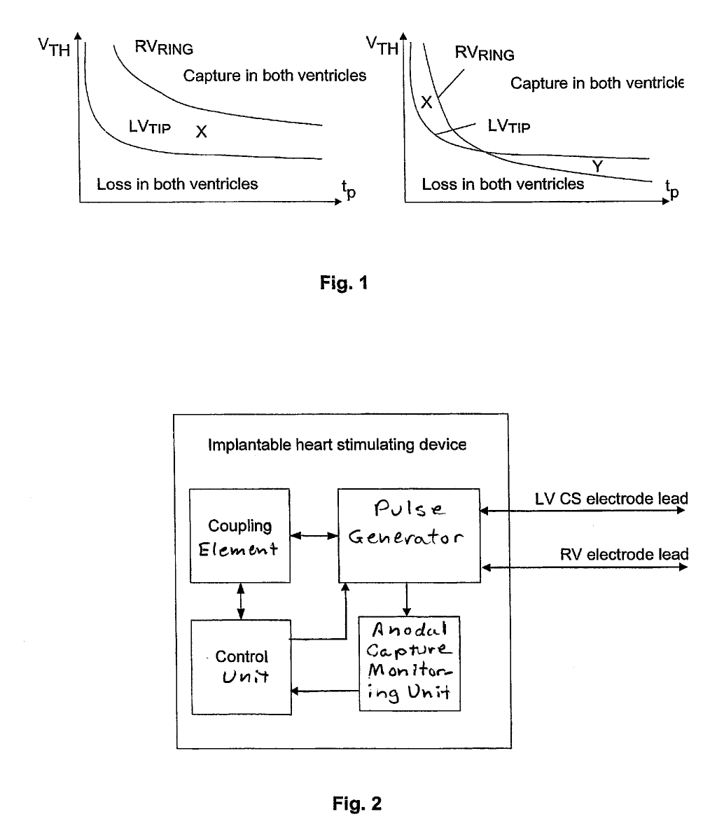

Thus, and according to a first preferred embodiment of the present invention, if anodal RV-ring stimulation is detected the LV pulse width is preferably adjusted so that LV-tip capture is obtained while anodal ring stimulation is not capturing the RV. This is schematically illustrated in FIG. 1 (see area “X” in FIG. 1).

FIG. 1 illustrates examples of unipolar strength-duration curves at anodal RV ring stimulation and cathodal LV tip stimulation. The Y-axis designates the stimulation threshold in volts, i.e. the amplitude of a stimulation pulse, and the X-axis designates the pulse width.

In the area “X” there is loss of capture in the right ventricle but capture in the left ventricle.

With references to the left graph there is loss of capture in both ventricles for stimulation pulses having the amplitudes and pulse widths below both curves, and capture in both ventricles above both curves. This graph illustrates a normal situation where the stimulation threshold is much higher on the anode (RV ring) than on the cathode (LV tip).

The right graph illustrates a situation where the two strength-duration curves are close to each other or even cross. In the area “Y” there is capture in the right ventricle but loss in the left ventricle. In “Y” it is not possible to only get capture in the left ventricle.

If the thresholds in RV and LV are close to each other (FIG. 1, right graph), it is, however, recommended not to use the RV ring as anode for the stimulation current. Instead the RV-ring in combination with the SVC-coil, RV-coil and/or the case is better to use. This is in accordance with a second preferred embodiment of the present invention, which will be further discussed below.

There are many different ways of detecting anodal (or bifocal) stimulation.

The above-mentioned U.S. Pat. No. 6,687,545 disclose a device where anodal stimulation is monitored by measuring the delay of the evoked response at the ring electrode, and if no delay is detected anodal stimulation is considered detected.

Other indications for anodal RV stimulation are:

Always ‘loss” at RV-stimulation:

Normally the left ventricle is stimulated before the right ventricle. Beat-to-beat capture verification is performed in the right ventricle being the last ventricle. If the system cannot detect capture in the last ventricle, despite the threshold searches postulate that it should be, it is an indication of anodal stimulation in the right ventricle before the actual RV stimulation. The reason for non-capture is that the myocardium in the right ventricle is refractory at the time of the RV stimulation due to bifocal capture at the time of the first stimulation in the left ventricle.

Always “capture” at RV-stimulation:

Again, normally the left ventricle is stimulated before the right ventricle. If the VV-delay is short an anodal RV-ring capture may be detected as capture from a stimulation pulse applied to the RV-ring. In this case the right ventricle heart tissue is refractory resulting in non-capture irrespectively of the level of the stimulation amplitude applied to the RV-ring electrode, because it is the LV stimulation pulse that stimulates the RV. Thus, if we still get capture in the RV (being the last ventricle) with zero pulse amplitude at the RV ring electrode it is an indication of anodal RV stimulation.

The threshold searches of the electrodes may be performed by temporarily switch mode to RV pacing (LV off). Then a threshold search with an anodal pacing pulse (e.g. the pacing may be between the case (negative) and the RV-ring (positive)) which will reveal the actual anodal threshold of the RV-ring. The same procedure is then performed in the LV (RV off) but with cathodic pacing pulse. If the two threshold values are close or even lower in the RV it is clear that it is difficult to find a stimulation pulse characteristic that avoids anodal stimulation of the RV-ring, instead other combinations of indifferent electrodes may be used according to the second preferred embodiment.

Still another way to detect anodal RV-ring stimulation is to analyze the IEGM in the RV at biventricular stimulation with the LV before the RV. By studying the evoked response (ER) morphology in the RV and changing the output level in the LV it is possible to find a point where the ER shifts in time from starting at LV-stim to RV-stim. A morphology change is likely to occur at this point. An obvious limitation with this method is that the RV threshold must be higher than the LV threshold.

When anodal ring stimulation in RV is detected and verified, actions have to be taken.

According to a first preferred embodiment threshold searches are performed at different pulse widths to find the optimal pulse width that gives the highest difference in cathodal LV threshold and anodal RV threshold. In this point the anodal RV strength duration curve must be above the cathodal LV threshold.

With references to FIG. 2, showing a block diagram of the inventive implantable heart stimulating device, the present invention will be further described.

The implantable heart stimulating device, according to a first embodiment, comprises a left ventricular (LV) coronary sinus (CS) electrode lead at least provided with a tip electrode, a right ventricular (RV) electrode lead at least provided with a ring electrode, a pulse generator connected to the leads and adapted to apply a stimulation pulse between the tip electrode and ring electrode, with the tip electrode being the cathode. The device also has an anodal stimulation monitor configured to monitor for and detect anodal stimulation at the right ventricular ring electrode subsequent to a stimulation. The monitor includes features necessary to perform normal evoked response detection. If anodal stimulation at the right ventricular ring electrode is detected, a threshold search is performed by varying the pulse width and/or pulse amplitude in order to identify stimulation pulse characteristics that avoid anodal stimulation at the ring electrode.

In addition the device has a control unit connected to the monitor and to the pulse generator. In response of a detected anodal stimulation the control unit generates and applies control signals to the pulse generator in order to initiate the threshold search.

As a result of the performed threshold search or searches, strength duration curves of the electrode(s) may be established and used to identify the stimulation pulse characteristics that avoid anodal stimulation at the ring electrode. In that case the identified stimulation pulse characteristics are chosen such that they are above the strength duration curve of the LV CS tip electrode but below the strength duration curve of the RV ring electrode, when inserted into a strength duration curve diagram (cf. the “X”-area in the left graph of FIG. 1).

According to a second preferred embodiment, if anodal RV stimulation is detected, a change or a proposal for a change is made of the anode for LV pacing from RV-ring to another electrode configuration. This electrode configuration includes the RV-ring and one or many of the case, RA-ring, LV-ring, RV-coil or SVC-coil (if available).

Also referring to FIG. 2 the implantable heart stimulating device has a left ventricular (LV) coronary sinus (CS) electrode lead at least provided with a tip electrode, a right ventricular (RV) electrode lead at least provided with a ring electrode, a pulse generator connected to the leads and adapted to apply a stimulation pulse between the tip electrode and ring electrode, with the tip electrode being the cathode. As in the first embodiment the device has a monitor adapted to monitor for and detect anodal stimulation at said right ventricular ring electrode subsequent to a stimulation. The monitor includes features necessary to perform normal evoked response detection.

If anodal stimulation at the right ventricular ring electrode is detected, at least one further electrode is arranged to function as indifferent electrode together with the ring electrode. This is achieved by electrically connecting the ring electrode to the at least one further electrode with a coupling element in response to control signals generated by the control unit.

The further electrode is arranged at any of the leads and may e.g. be a coil electrode at the right or left ventricular electrode lead and/or at the housing of the device.

Thus, the monitoring of anodal stimulation may e.g. be performed by measuring the delay of the evoked response at the ring electrode, and if no delay is detected anodal stimulation is considered detected. This is further described above in connection with the prior art document U.S. Pat. No. 6,687,545.

Other ways of monitoring and detecting anodal stimulation are described above.

In the following some other aspects of anodal stimulation are briefly discussed.

If the anodal RV-ring threshold is lower than the LV threshold and the patient benefits from a short VV-delay the RV stimulation can just be turned off. Instead every ventricular stimulation will stimulate both LV and RV simultaneously which saves energy. Evoked response detectors are still active in both ventricles to verify capture. If a loss occurs in either ventricle the pacing amplitude is increased as usual. If that does not help to get anodal capture in RV the system switches the pacing mode to dual ventricular pulse (preferably not with RV ring as anode).

The actions mentioned above can either be performed automatically by the device or be performed by the healthcare personnel at next follow up at the healthcare centre. The alert to change setting may be communicated to the physician via the programmer.

If anodal stimulation suddenly appears it is recommended to perform threshold search also in the right ventricle since the cathodic threshold may also have changed.

Although modifications and changes may be suggested by those skilled in the art, it is the intention of the inventors to embody within the patent warranted heron all changes and modifications as reasonably and properly come within the scope of their contribution to the art.

We claim as our invention:—therefor

1-10. (canceled)

11. An implantable heart stimulating device comprising:
a left ventricular coronary sinus electrode lead carrying a tip electrode;
a right ventricular electrode lead carrying a ring electrode;
a pulse generator connected to said leads and configured to apply stimulation pulses between said tip electrode and said ring electrode, with the tip electrode serving as the cathode;
a monitoring unit configured to monitor for and detect anodal capture at said ring electrode subsequent to application of a stimulation pulse from said pulse generator; and
a control unit connected to said monitoring unit and configured, if anodal capture at said ring electrode is detected, to initiate a stimulation energy threshold search by varying at least one of a pulse width and a pulse amplitude of said stimulation pulses to identify at least one stimulation pulse characteristic that avoids said anodal capture at said ring electrode.

12. An implantable heart stimulating device as claimed in claim 11 comprising a memory, accessible by said control unit, containing strength duration curves for said electrodes, and wherein said control unit identifies said stimulation pulse characteristics that avoid said anodal capture at said ring electrode using said strength duration curves.

13. An implantable heart stimulating device as claimed in claim 12 wherein said control unit identifies stimulation pulse characteristics that are above a respective strength duration curve for said left ventricular coronary sinus electrode and below a respective strength duration curve of said right ventricular ring electrode.

14. An implantable heart stimulating device as claimed in claim 11 wherein said monitoring unit is configured to monitor anodal capture by measuring a delay of an evoked response at said ring electrode, with anodal capture being detected if no delay is detected.

15. An implantable heart stimulating device comprising:
a left ventricular coronary sinus electrode lead carrying a tip electrode;
a right ventricular electrode lead carrying a ring electrode;
a pulse generator connected to said leads and configured to apply stimulation pulses between said tip electrode and said ring electrode, with the tip electrode serving as the cathode;
a further electrode configured for implantation together with said ring electrode and said tip electrode;
a monitoring unit configured to monitor for and detect anodal capture at said ring electrode subsequent to application of a stimulation pulse from said pulse generator; and
said stimulation pulse generator, if anodal capture at said right ventricular ring electrode is detected, being configured to employ said further electrode to function as an indifferent electrode in combination with said ring electrode.

16. An implantable heart stimulating device as claimed in claim 15 wherein said further electrode is carried by one of said left ventricular coronary sinus electrode lead or said right ventricular electrode lead.

17. An implantable heart stimulating device as claimed in claim 15 comprising a housing containing said pulse generator, and wherein said further electrode is carried at said housing.

18. An implantable heart stimulating device as claimed in claim 15 wherein said further electrode is a coil electrode carried by said right ventricular electrode lead.

19. An implantable heart stimulating device as claimed in claim 15 comprising a control unit connected to said monitoring unit and a coupling element operable by said control unit, said control unit being configured to operate said coupling element to electrically connect said ring electrode and said further electrode upon detection of said anodal capture at said ring electrode.

20. An implantable heart stimulating device as claimed in claim 15 wherein said monitoring unit is configured to monitor anodal capture by measuring a delay of an evoked response at said ring electrode, with anodal capture being detected if no delay is detected.
